(19) United States
(12) Patent Application Publication
(10) Pub. No.: US 2009/0270901 A1
(43) Pub. Date: Oct. 29, 2009

(54) Degradable detachment mechanisms for implantable devices

(75) Inventors: Brian Kelleher, Del Mar, CA (US); Matt Yurek, San Diego, CA (US); Corbett Stone, San Diego, CA (US)

Correspondence Address: ROBINS & PASTERNAK, 1731 EMBARCADERO ROAD, SUITE 230, PALO ALTO, CA, 94303, US

(73) Assignee: Boston Scientific Scimed, Inc.

(21) Appl. No.: 12/290,652

(22) Filed: Oct. 30, 2008

Related U.S. Application Data
(60) Provisional application No. 61/000,973, filed on Oct. 30, 2007.

(51) Int. Cl. A61M 37/00

(52) U.S. Cl. 606/191

(57) ABSTRACT

Described herein are degradable detachment mechanisms for implantable devices and assemblies comprising these devices. Also provided are methods of using the detachment mechanisms and assemblies.

### CROSS-REFERENCE TO RELATED APPLICATIONS

The present application claims the benefit of U.S. Provisional Application No. 61/000,973, filed Oct. 30, 2007, the disclosure of which is hereby incorporated by reference in its entirety.

### FIELD OF THE INVENTION

This invention relates to degradable detachment mechanisms for implantable devices.

### BACKGROUND

An aneurysm is a dilation of a blood vessel that poses a risk to health from the potential for rupture, clotting, or dissecting. Rupture of an aneurysm in the brain causes stroke, and rupture of an aneurysm in the abdomen causes shock. Cerebral aneurysms are usually detected in patients as the result of a seizure or hemorrhage and can result in significant morbidity or mortality.

There are a variety of materials and devices which have been used for treatment of aneurysms, including platinum and stainless steel microcoils, polyvinyl alcohol sponges (Ivalone), and other mechanical devices. For example, vaso-occlusion devices are surgical implements or implants that are placed within the vasculature of the human body, typically via a catheter, either to block the flow of blood through a vessel making up that portion of the vasculature through the formation of an embolus or to form such an embolus within an aneurysm stemming from the vessel. One widely used vaso-occlusive device is a helical wire coil having windings that may be dimensioned to engage the walls of the vessels. (See, e.g., U.S. Pat. No. 4,994,069 to Ritchart et al.). Variations of such devices include polymeric coatings or attached polymeric filaments have also been described. See, e.g., U.S. Pat. Nos. 5,226,911; 5,935,145; 6,033,423; 6,280,457; 6,287,318; and 6,299,627. In addition, coil designs including stretch-resistant members that run through the lumen of the helical vaso-occlusive coil have also been described. See, e.g., U.S. Pat. Nos. 5,582,619; 5,833,705; 5,853,418; 6,004,338; 6,013,084; 6,179,857; and 6,193,728.

Typically, implantable devices include a detachment mechanism in order to be released from the deployment mechanism (e.g., attached wire). Several classes of techniques have been developed to enable more accurate placement of implantable devices within a vessel. One class involves the use of electrolytic means to detach the vasoocclusive member from the pusher. Electrolytic coil detachment is disclosed in U.S. Pat. Nos. 5,122,136; 5,354,295; 6,620,152; 6,425,893; and 5,976,131, all to Guglielmi et al., describe electrolytically detachable embolic devices. U.S. Pat. No. 6,623,493 describes vaso-occlusive member assembly with multiple detaching points. U.S. Pat. Nos. 6,589,236 and 6,409,721 describe assemblies containing an electrolytically severable joint. The coil is bonded via a metal-to-metal joint to the distal end of the pusher. The pusher and coil are made of dissimilar metals. The coil-carrying pusher is advanced through the catheter to the site and a small electrical current is passed through the pusher-coil assembly. The current causes the joint between the pusher and the coil to be severed via electrolysis. The pusher may then be retracted leaving the detached coil at an exact position within the vessel. Since no significant mechanical force is applied to the coil during electrolytic detachment, highly accurate coil placement is readily achieved. In addition, the electric current may facilitate thrombus formation at the coil site. The disadvantage of this method is that the electrolytic release of the coil may require a period of time that may inhibit rapid detachment of the coil from the pusher.

Other forms of energy are also used to sever sacrificial joints that connect pusher and vasoocclusive member apparatus. Sacrificial connection member, preferably made from polyvinylacetate (PVA), resins, or shape memory alloys, can be used to join a conductive wire to a detention member. See, U.S. Pat. Nos. 5,759,161 and 5,846,210. Upon heating by a monopolar high frequency current, the sacrificial connection member melts, severing the wire from the detention member.

U.S. Pat. No. 5,944,733 describes application of radiofrequency energy to sever a thermoplastic joint and U.S. Pat. No. 6,743,251 describes detachment joints that are severed by the application of low frequency energy or direct current. U.S. Pat. No. 6,346,091 describes a wire detachment junction that is severed by application of vibrational energy.

In U.S. Pat. No. 4,735,201 to O'Reilly, an optical fiber is enclosed within a catheter and connected to a metallic tip on its distal end by a layer of hot-melt adhesive. The proximal end of the optical fiber is connected to a laser energy source. When endovascularly introduced into an aneurysm, laser energy is applied to the optical fiber, heating the metallic tip so as to cauterize the immediately surrounding tissue. The layer of hot-melt adhesive serving as the bonding material for the optical fiber and metallic tip is melted during this lasing, but the integrity of the interface is maintained by application of back pressure on the catheter by the physician. When it is apparent that the proper therapeutic effect has been accomplished, another pulse of laser energy is then applied to once again melt the hot-melt adhesive, but upon this reheating the optical fiber and catheter are withdrawn by the physician, leaving the metallic tip in the aneurysm as a permanent plug.

Other methods for placing implantable devices within the vasculature utilize heat releasable bonds that can be detached by using laser energy (see, U.S. Pat. No. 5,108,407). EP 0 992 220 describes an embolic coil placement system which includes conductive wires running through the delivery member. When these wires generate sufficient heat, they are able to sever the link between the embolic coil and the delivery wires. Further, U.S. Pat. No. 6,113,622 describes the use of fluid pressure (e.g., hydraulics) to detach an embolic coil.

The above documents relate to detachment mechanisms that are sacrificial joints. Thus, there remains a need for degradable detachment mechanisms that contact the implant and hold it in place until they are degraded.

### SUMMARY

Described herein are detachment mechanisms made of a material which can be rapidly degraded (e.g., by application of energy and/or upon contact a solvent or fluid). Unlike previously described detachment junctions which take the form of sacrificial joints distal to the implantable device, the degradable material of the detachment mechanisms described herein surrounds at least a portion of the proximal end of the implantable device and holds the device in place within the deployment device (e.g., catheter). When the detachment mechanism is degraded (e.g., fractured, fluidized, dissolved, etc.), the material no longer holds the device in the catheter and the implant is released.

In certain aspects, disclosed herein is an assembly comprising: an implantable device having a proximal region and a distal region, and a detachment mechanism comprising a degradable material, wherein the detachment mechanism surrounds at least portion of the proximal region of the implantable device and secures the implantable device to a delivery device when the material is not degraded. The degradable material may be, for example, salt, sugar, glass, one or more polymers (e.g., poly-L-lactic acid (PLLA), polyglycolic acid (PGA), polyvinyl alcohol (PVA) and/or combinations thereof), lipids, crystal structures, tetrahedrons (e.g., tightly packed tetrahedrons), and/or combinations thereof.

In any of the assemblies described herein, the assembly may further comprise a degrading element that degrades the degradable material. The degrading element may be any means that degrades the degradable material and, in certain embodiments, the degrading element (or degrading means) contacts the degradable material. The degrading element (means) may be, for example, a source of energy (e.g., electromagnetic radiation, thermal energy, electrical energy, vibrational energy (e.g., ultrasonic energy), and/or combinations thereof). In certain embodiments, the energy is electromagnetic radiation and is selected from the group consisting of radio waves, microwaves, terahertz radiation, infrared radiation, visible light, ultraviolet radiation, X-rays, gamma rays and combinations thereof. In other embodiments, the degrading element or degrading means comprises a fluid (e.g., water, saline, blood or combinations thereof).

In any of the assemblies described herein, the implantable device may comprise a vaso-occlusive device, for example a vaso-occlusive coil or a tubular braid. Furthermore, any of the assemblies described herein may further comprise a delivery device (e.g., catheter, microcatheter, etc.).

In another aspect, described herein is a method of occluding a body cavity, the method comprising introducing one or more of any of the implantable assemblies described herein into the body cavity. In certain embodiments, the body cavity is an aneurysm.

These and other embodiments will readily occur to those of skill in the art in light of the disclosure herein.

### BRIEF DESCRIPTION OF THE FIGURES

FIG. 1 is a side view of an exemplary assembly comprising a degradable detachment mechanism as described herein.

FIG. 2, panels A and B, are side views of an exemplary degradable detachment mechanism comprising a solid structure (FIG. 2A) that is fluidized upon application of energy and/or other materials (FIG. 2B).

### DETAILED DESCRIPTION

Detachment mechanisms for implantable devices and assemblies comprising these detachment mechanisms are described. The detachment mechanisms described herein find use in deploying vascular and neurovascular implants and are particularly useful in treating aneurysms, for example small-diameter, curved or otherwise difficult to access vasculature, for example aneurysms, such as cerebral aneurysms. Methods of making and using these detachment mechanisms and assemblies are also described.

All publications, patents and patent applications cited herein, whether above or below, are hereby incorporated by reference in their entirety.

It must be noted that, as used in this specification and the appended claims, the singular forms “a”, “an”, and “the” include plural referents unless the content clearly dictates otherwise.

The detachment mechanisms described herein that allow for rapid and precise detachment of an implantable device upon operator-induced degradation of the material surrounding at least a portion of the implantable device.

Any degradable material can be used in the detachment mechanisms described herein, including naturally occurring materials, synthetic materials or combinations of natural and synthetic materials. Non-limiting examples of suitable degradable materials include salt, sugar, glass, polymers (e.g., poly-L-lactic acid (PLLA), polyglycolic acid (PGA), polyvinyl alcohol (PVA), as well as other degradable polymers known to those of skill in the art), lipids (e.g., cholesterol), other crystal structures and/or tetrahedron materials.

In certain embodiments, the detachment mechanisms are degraded by the application of energy. Examples of suitable forms of energy include, but are not limited to, electromagnetic radiation (e.g., radio waves, microwaves, terahertz radiation, infrared radiation, visible light, ultraviolet radiation, X-rays and gamma rays), heat (thermal) energy, electrical energy, vibrational energy (e.g., sonic or ultrasonic) and combinations thereof.

Alternatively, the detachment mechanism may be degraded upon exposure to one or more substances (e.g., fluids, solvents, gels, etc.). In certain embodiments, a fluid is used to degrade the detachment mechanism. Preferably, the fluid is biocompatible, for example, saline, water, blood may be used to dissolve detachment mechanisms comprising sugar, salt or the like. While the material that degrades the detachment element may be within the patient (e.g., blood from the vasculature), it is preferred that the operator introduce the fluid through the deployment device so that release of the implantable device is controlled by the operator and occurs rapidly into the desired location.

Delivery mechanisms (e.g., catheter or delivery tube) that allow for energy and/or materials (e.g., fluids) to be transmitted to the detachment mechanism include, for example, multi-lumen catheters for transmitting fluids and catheters comprising energy conductors (e.g., electrodes or heat conductors) in the side-walls are known to those of skill in the art. See, e.g., U.S. Pat. Nos. 6,059,779 and 7,020,516. Conductors of the degradation substance may also be transmitted through the lumen of the delivery mechanism. For example, bi-polar electrodes and/or anodes alone or twisted with a core wire cathode can also be used to supply current to the degradable detachment mechanism. The conductive element may include a polymer jacket/liner to insulate the conductors and/or reduce friction during advancement. Thus, the energy or other substances that induce degradation can be from the proximal end of the delivery device to the degradable detachment mechanism via such conductors.

Depicted in the appended drawings are exemplary embodiments of the present invention in which the implantable device is depicted as an embolic device. It will be appreciated that the drawings are for purposes of illustration only and that other implantable devices can be used in place of embolic devices, for example, stents, filters, and the like. Furthermore, although depicted in the Figures as embolic coils, the embolic devices may be of a variety of shapes or configuration including, but not limited to, braids, wires, knits, woven structures, tubes (e.g., perforated or slotted tubes), injection-molded devices and the like. See, e.g., U.S. Pat. No. 6,533,801 and International Patent Publication WO 02/096273. It will also be appreciated that the assemblies can have various configurations as long as the required flexibility is present.

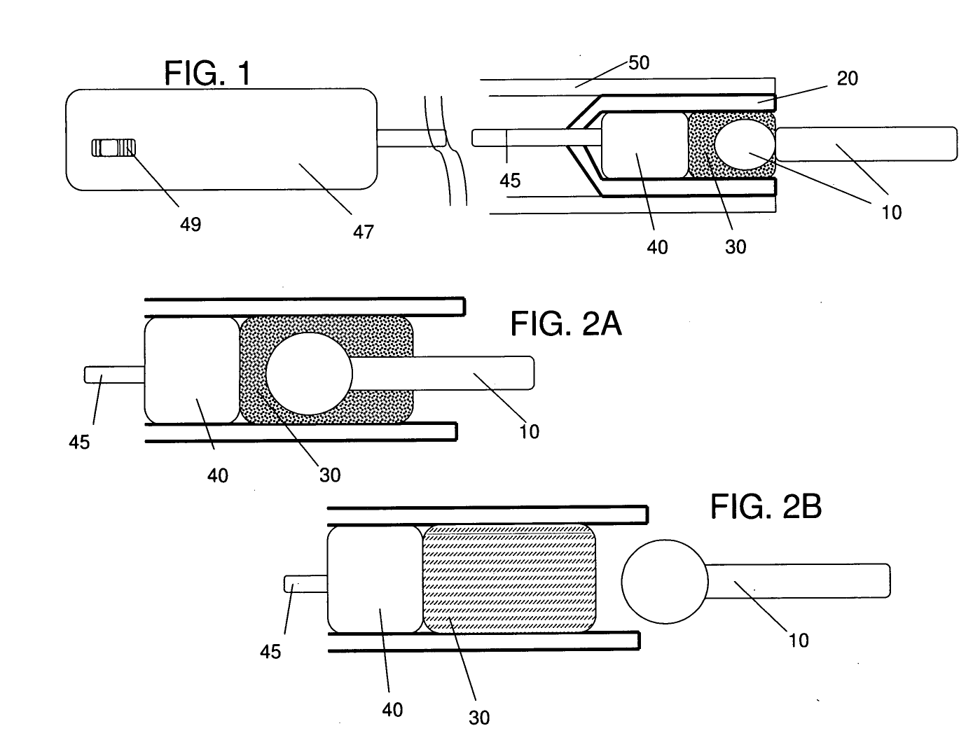

FIG. 1 is a side and view of an exemplary assembly comprising a degradable detachment mechanism as described herein. In particular, the implantable coil 10 is shown held in place within a deployment catheter 50 by the degradable detachment mechanism 30 in the non-degraded (solid) form. The detachment mechanism 30 may comprise materials that are degraded by application of different forms of energy or by one or more solvents or fluids. Also shown is element 40 for degrading the detachment mechanism 30 via application of energy or other degradation-inducing materials. The degradation-inducing element 40 may transmit energy or other substances (e.g., fluids) from an energy source or reservoir 47 via a conductor 45.

Conductor element 45 will be any configuration and material that allows for delivery of the degrading input. For example, in the case of energy, the conductor element may comprise an conductive material such as stainless steel, platinum, gold, etc. In cases where the detachment mechanism is degraded by solvents of fluids, conductor element 45 may comprise a lumen into which the operator can inject the fluid or solvent so that is fills the transmitter element 40 and degrades the detachment mechanism 30. One or more conductor elements may be present. Furthermore, although shown in the Figures as positioned in the lumen of the delivery device, it will be apparent that the conductor element 45 can be positioned in the sidewalls of the selected delivery device.

The reservoir or energy source 47 may include one or more actuators 49 which allow the operator to input the degrading energy or substance to degrade the detachment mechanism 30 when deployed of the implant 10 is desired.

A sleeve or collar 20 of any configuration may be used to encase the proximal end of the implant 10, the detachment mechanism 30 and the element that supplies the degrading energy or degrading substance 40.

FIG. 2A shows an exemplary degradable detachment mechanism 30 comprising tightly packed tetrahedrons which anchor the implantable coil 10 within the delivery mechanism. FIG. 2B illustrates how, upon application of vibrational energy by the operator which energy is transmitted to the device by elements 40, 45, the detachment mechanism 30 is degraded (fluidized) and the implantable coil 10 deployed.

With regard to particular materials used in the implantable devices and assemblies of the invention, it is to be understood that the implantable devices or assemblies may be made of a variety of materials, including but not limited to metals, polymers and combinations thereof, including but not limited to, stainless steel, platinum, kevlar, PET, catbothane, cyanoacrylate, epoxy, poly(ethyleneterephthalate) (PET), polytetrafluoroethylene (Teflon™), polypropylene, polyimide polyethylene, polyglycolic acid, polylactic acid, nylon, polyester, fluoropolymer, and copolymers or combinations thereof. See, e.g., U.S. Pat. Nos. 6,585,754 and 6,280,457 for a description of various polymers. Different components of the devices and assemblies may be made of different materials.

In embodiments in which the implantable device comprises an embolic coil, the main coil may be a coiled and/or braided structure comprising one or more metals or metal alloys, for example, Platinum Group metals, especially platinum, rhodium, palladium, rhenium, as well as tungsten, gold, silver, tantalum, stainless steel and alloys of these metals. Preferably, the comprises a material that maintains its shape despite being subjected to high stress, for example, “super-elastic alloys” such as nickel/titanium alloys (48-58 atomic % nickel and optionally containing modest amounts of iron); copper/zinc alloys (38-42 weight % zinc); copper/zinc alloys containing 1-10 weight % of beryllium, silicon, tin, aluminum, or gallium; or nickel/aluminum alloys (36-38 atomic % aluminum). Particularly preferred are the alloys described in U.S. Pat. Nos. 3,174,851; 3,351,463; and 3,753,700. Especially preferred is the titanium/nickel alloy known as “nitinol.” The main coil may also comprise a shape memory polymer such as those described in International Publication WO 03/51444. The implantable device is preferably electrically insulated, for example, by coating a metallic coil (e.g., stainless steel, platinum) with one or more electrically insulating materials, for example one or more polymers such as polyimide.

The implantable device may also change shape upon release from the deployment mechanism (e.g., pusher wire), for example change from a linear form to a relaxed, three-dimensional configuration upon deployment.

The devices described herein may also comprise additional components, such as co-solvents, plasticizers, coalescing solvents, bioactive agents, antimicrobial agents, antithrombogenic agents (e.g., heparin), antibiotics, pigments, radiopacifiers and/or ion conductors which may be coated using any suitable method or may be incorporated into the element(s) during production. See, e.g., U.S. Pat. No. 6,585,754 and WO 02/051460, U.S. Pat. No. 6,280,457. The additional components can be coated onto the device and/or can be placed in the vessel prior to, concurrently or after placement of one or more devices as described herein.

The devices described herein are often introduced into a selected site using the procedure outlined below. This procedure may be used in treating a variety of maladies. For instance in the treatment of an aneurysm, the aneurysm itself will be filled (partially or fully) with the compositions described herein.

Conventional catheter insertion and navigational techniques involving guidewires or flow-directed devices may be used to access the site with a catheter. The mechanism will be such as to be capable of being advanced entirely through the catheter to place vaso-occlusive device at the target site but yet with a sufficient portion of the distal end of the delivery mechanism protruding from the distal end of the catheter to enable detachment of the implantable vaso-occlusive device. For use in peripheral or neural surgeries, the delivery mechanism will normally be about 100-200 cm in length, more normally 130-180 cm in length. The diameter of the delivery mechanism is usually in the range of 0.25 to about 0.90 mm. Briefly, occlusive devices (and/or additional components) described herein are typically loaded into a carrier for introduction into the delivery catheter and introduced to the chosen site using the procedure outlined below. This procedure may be used in treating a variety of maladies. For instance, in treatment of an aneurysm, the aneurysm itself may be filled with the embolics (e.g. vaso-occlusive members and/or liquid embolics and bioactive materials) which cause formation of an emboli and, at some later time, is at least partially replaced by neovascularized collagenous material formed around the implanted vaso-occlusive devices.

A selected site is reached through the vascular system using a collection of specifically chosen catheters and/or guide wires. It is clear that should the site be in a remote site, e.g., in the brain, methods of reaching this site are somewhat limited. One widely accepted procedure is found in U.S. Pat. No. 4,994,069 to Ritchart, et al. It utilizes a fine endovascular catheter such as is found in U.S. Pat. No. 4,739,768, to Engelson. First of all, a large catheter is introduced through an entry site in the vasculature. Typically, this would be through a femoral artery in the groin. Other entry sites sometimes chosen are found in the neck and are in general well known by physicians who practice this type of medicine. Once the introducer is in place, a guiding catheter is then used to provide a safe passageway from the entry site to a region near the site to be treated. For instance, in treating a site in the human brain, a guiding catheter would be chosen which would extend from the entry site at the femoral artery, up through the large arteries extending to the heart, around the heart through the aortic arch, and downstream through one of the arteries extending from the upper side of the aorta. A guidewire and neurovascular catheter such as that described in the Engelson patent are then placed through the guiding catheter. Once the distal end of the catheter is positioned at the site, often by locating its distal end through the use of radiopaque marker material and fluoroscopy, the catheter is cleared and/or flushed with an electrolyte solution.

Once the selected site has been reached, the vaso-occlusive device is extruded using a pusher-detachment mechanism as described herein and released in the desired position of the selected site.

Modifications of the procedures and assemblies described above, and the methods of using them in keeping with this disclosure will be apparent to those having skill in this mechanical and surgical art. These variations are intended to be within the scope of the claims that follow.

What is claimed is:

1. An assembly comprising:
an implantable device having a proximal region and a distal region, and
a detachment mechanism comprising a degradable material, wherein the detachment mechanism surrounds at least portion of the proximal region of the implantable device and secures the implantable device to a delivery device when the material is not degraded.

2. The assembly of claim 1, wherein the degradable material is selected from the group consisting of salt, sugar, glass, one or more polymers, lipids, crystal structures, tetrahedrons, and combinations thereof.

3. The assembly of claim 2, wherein the degradable material comprises a polymer selected from the group consisting of poly-L-lactic acid (PLLA), polyglycolic acid (PGA), polyvinyl alcohol (PVA) and combinations thereof.

4. The assembly of claim 2, wherein the degradable material comprises tightly packed tetrahedrons.

5. The assembly of claim 1, further comprising a means for degrading the degradable material.

6. The assembly of claim 5, wherein the means for degrading the degradable material contacts the degradable material.

7. The assembly of claim 5, wherein the means for degrading the degradable material comprises a source of energy.

8. The assembly of claim 7, wherein the energy is selected from the group consisting of electromagnetic radiation, thermal energy, electrical energy, vibrational energy, and combinations thereof.

9. The assembly of claim 8, wherein the energy is electromagnetic radiation and the electromagnetic radiation is selected from the group consisting of radio waves, microwaves, terahertz radiation, infrared radiation, visible light, ultraviolet radiation, X-rays, gamma rays and combinations thereof.

10. The assembly of claim 8, wherein the vibrational energy is ultrasonic energy.

11. The assembly of claim 5, wherein the means for degrading the degradable material comprises a fluid.

12. The assembly of claim 11, wherein the fluid is selected from the group consisting of water, saline, blood or combinations thereof.

13. The assembly of claim 1, wherein the implantable device comprises a vaso-occlusive device.

14. The assembly of claim 13, wherein the vaso-occlusive device is a coil or a tubular braid.

15. The assembly of claim 1, further comprising a delivery device.

16. The assembly of claim 15, wherein the delivery device comprises a catheter.

17. A method of occluding a body cavity comprising introducing an implantable assembly according to claim 1 into the body cavity.

18. The method of claim 17, wherein the body cavity is an aneurysm.
